(19) United States
(12) Patent Application Publication
(10) Pub. No.: US 2024/0027435 A1
(43) Pub. Date: Jan. 25, 2024

(54) METHOD AND KIT FOR LABELING EUKARYOTIC CELLS FROM A MULTICELLULAR ORGANISM USING MODIFIED MONOSACCHARIDE COMPOUNDS AND PHARMACEUTICAL COMPOSITION COMPRISING SUCH CELLS

(71) Applicant: THERAONCO, MARSEILLE CEDEX 9 (FR)

(72) Inventor: Sam DUKAN, MARSEILLE (FR)

(21) Appl. No.: 18/021,761

(22) PCT Filed: Aug. 18, 2021
(86) PCT No.: PCT/EP2021/072963

§ 371 Date: Feb. 16, 2023

(30) Foreign Application Priority Data
Aug. 19, 2020 (EP) 20305935.7

(51) Int. Cl. G01N 33/533; G01N 33/574; A61K 31/7004; A61P 35/00

(52) U.S. Cl. CPC G01N 33/533; G01N 33/574; A61K 31/7004; A61P 35/00

(57) ABSTRACT

Methods which use click-chemistry modified monosaccharide compounds of the pentose phosphate pathway, e.g. ribose, ribulose, arabinitol, xylulose, xylose or xylitol, for labeling and/or detecting an eukaryotic cell from a multicellular organism. It also relates to such modified monosaccharide compounds implemented in methods for identifying or isolating cancer cells, diagnosing a cancer or for cell therapy.

### FIELD OF THE INVENTION

The present invention relates to the medicinal field, in particular of oncology. It relates to modified monosaccharide compounds implemented in methods for labeling and/or detecting and/or targeting an eukaryotic cell from a multicellular organism. It also relates to such modified monosaccharide compounds implemented in methods for identifying or isolating cancer cells, diagnosing a cancer or for cell therapy.

### BACKGROUND OF THE INVENTION

Carbohydrates are important as signaling molecules and for cellular recognition events. Indeed, they can produce multivalent interactions with carbohydrate recognition proteins (CRPs) and be used as probes of living organisms. Carbohydrates thus present many opportunities in disease diagnosis and therapy. As a consequence, the development of carbohydrate-based bioactive compounds and sensors has become an active research area. An effective and modular synthetic approach to prepare functional carbohydrates derivatives is click chemistry. Some carbohydrate derivatives prepared by CuAAC (Cu-catalyzed azide-alkyne 1,3-dipolar cycloaddition) click chemistry for therapy and diagnosis are reported in the review from He et al. (Carbohydrate Research 429 (2016) 1-22). The copper-free click chemistry using the strain-promoted azide-alkyne cycloaddition reaction has also been described for cancer cells. Specific sugars presenting clickable groups (e.g., triacetyl N-azidoacetylmannosamine (Ac₃ManNAz) analogs) are metabolized by cancer cells due to specific overexpressing enzyme-cleavable sugar analogs. Said sugars are thus metabolized and incorporated into cancer cell membranes specifically.

Even if a variety of carbohydrate/monosaccharide click chemistry has been described for therapy and diagnosis, these click chemistry methods and the compounds used therein appear to be cytotoxic and/or not selective to specific cells, such as selective to cancer cells. Cytotoxicity of these compounds implies to use low non-toxic concentrations which render them less effective.

WO2013/1077559 describes modified monosaccharide compounds, such as the specific compound 8-azido-3,8-dideoxy-D-manno-octulosonic acid (also called herein “KDO-N₃) in methods for labeling specifically living microorganisms. The labeled living microorganisms have been limited to unicellular prokaryotic microorganisms (bacteria).

WO2016/177712 also describes modified monosaccharide compounds, such as the specific compound 5-azido-5-deoxy-D-arabinofuranose (also called herein “Ara-N₃”) in methods for labeling specifically living microorganisms. The labeled living microorganisms have been limited to unicellular prokaryotic microorganisms (bacteria) and unicellular eukaryotic microorganisms (yeast, fungi and amoebas).

However, so far, no explanation can be provided on how and where said monosaccharide compounds are assimilated by cellular membranes of said microorganisms.

There is a constant need to find and develop new candidates, especially for labeling and detecting eukaryotic cell from multicellular organisms, to provide methods for identifying or isolating cancer cells, and to provide methods of diagnosis or cell therapy, particularly in the cancer field.

### SUMMARY OF THE INVENTION

The present invention is based on monosaccharides compounds of the pentose phosphate pathway, excluding arabinose, wherein said compounds further comprise a reactive group X allowing to covalently link a further compound, such as a label or an anti-cancer drug or particles comprising an anti-cancer drug, via a click chemistry reaction. The formed conjugates may thus be implemented in methods for labeling or detecting eukaryotic cells from a multicellular organism, for identifying or isolating cancer cells, or for treating a cancer.

More particularly, the inventors have found that assimilation of such modified monosaccharides occurs with eukaryotic cell from a multicellular organism. They have also found that such assimilation in tumoral eukaryotic cells was different from non-tumoral eukaryotic cells and also more important compared to non-tumoral cells (in particular for bladder, blood, skin, pancreas, brain, liver, kidney, lung, muscle, lymphocyte, prostate, stomach, breast cancer compared to non-cancer cells). Therefore, the monosaccharides compounds can also be used as cancer probes and markers which can be useful for identifying, isolating or targeting cancer cells, and/or for diagnosing a cancer in a subject.

Accordingly, an aspect of the present invention is a method, preferably an in vitro method, for labeling or detecting or targeting an eukaryotic cell from a multicellular organism, the method comprising the steps of:
a) contacting a sample comprising eukaryotic cells with at least one modified monosaccharide compound of the pentose phosphate pathway, excluding arabinose;
b) contacting the sample of step (a) with a compound bearing a first reactive group, optionally in presence of copper; and
c) optionally detecting the compound of step (b) bond to the monosaccharide of step (a), as to detect eukaryotic cells;
   wherein said at least one modified monosaccharide compound of the pentose phosphate pathway comprise a reactive group X, called a second reactive group, the first and the second reactive groups are able to react together in a click chemistry reaction, as to obtain the compound of step (b) bond to the said monosaccharide. In a preferred embodiment, the first reactive group is an alkyne group and the second reactive group X is an azido group (—N₃).

A further aspect of the invention is a kit for implementing the method for labeling, detecting or targeting an eukaryotic cell as defined herein, comprising:
a modified monosaccharide compound of the pentose phosphate pathway, excluding arabinose, wherein said monosaccharide comprises a reactive group X, called a second reactive group, as defined herein, and
a compound bearing a first reactive group, as defined herein.

A further aspect of the invention is a method for identifying or isolating cancer cells or for diagnosing a cancer in a subject, comprising implementing a method for labeling, detecting, or targeting an eukaryotic cell from a multicellular organism, as defined herein, from said subject, preferably in a biological sample from said subject.

A further aspect of the invention is a composition comprising an eukaryotic cell presenting on its surface at least one modified monosaccharide compound of the pentose phosphate pathway (excluding arabinose), operably linked or not to an anti-cancer drug or to particles comprising at least one anti-cancer drug. Another aspect is a pharmaceutical composition comprising such cell. Another aspect is such pharmaceutical composition for use in treating a cancer, especially by cell-based therapy. Another aspect is such pharmaceutical composition for use in diagnosing a cancer.

Another aspect of the invention is a method for treating a cancer in a subject in need thereof, comprising administering to said subject an efficient amount of a composition, as defined herein, comprising an eukaryotic cell presenting on its surface at least one modified monosaccharide compound of the pentose phosphate pathway (excluding arabinose), as defined herein, operably linked to an anti-cancer drug or to particles comprising at least one anti-cancer drug.

A further aspect of the invention is a use of a modified monosaccharide compound of the pentose phosphate pathway (excluding arabinose), as defined herein for medical imaging or diagnosis, preferably for diagnosing a cancer, optionally with a compound bearing a first reactive group.

### DETAILED DESCRIPTION OF THE INVENTION

### Definitions

According to the present invention, the terms below have the following meanings:

A “multicellular organism” comprises any organism comprising more than one cell. A multicellular organism derives from or is, for instance, a plant or an animal, preferably a mammal, more preferably a human.

As used herein, the terms “patient” and “subject” can be used interchangeably and include both humans and animals, more specifically humans.

An “eukaryotic cell from a multicellular organism” is a cell having a nucleus within membranes, unlike prokaryotes, and coming from a multicellular organism, such as animal or plant cells. Animals and plant cells are the most familiar eukaryotes cells from a multicellular organism. In the context of the present invention, “eukaryotic cells” include cancer cells or normal cells (or non-tumoral and tumoral cells).

Cancer cells are cells that divide relentlessly, forming solid tumors or flooding the blood with abnormal cells. It can thus be a solid cancer or a hematopoietic cancer, such as lymphoma or leukemia.

The term “cancer” or “tumor”, as used herein, refers to the presence of cells possessing characteristics typical of cancer-causing cells, such as uncontrolled proliferation, immortality, metastatic potential, rapid growth and proliferation rate, and certain characteristic morphological features. This term refers to any type of malignancy (primary or metastases). Typical cancers are solid or hematopoietic cancers such as breast, brain, stomach, liver, skin, prostate, pancreatic, oesophageal, sarcoma, ovarian, endometrium, bladder, cervix uteri, rectum, colon, renal, lung or ORL cancers, paediatric tumors (neuroblastoma, glioblastoma multiforme), lymphoma, carcinoma, glioblastoma, hepatoblastoma, leukemia, myeloma, seminoma, Hodgkin or malignant hemopathies. According to the invention, the term “comprise(s)” or “comprising” (and other comparable terms, e.g., “containing,” and “including”) is “open-ended” and can be generally interpreted such that all of the specifically mentioned features and any optional, additional and unspecified features are included. According to specific embodiments, it can also be interpreted as the phrase “consisting essentially of” where the specified features and any optional, additional and unspecified features that do not materially affect the basic and novel characteristic(s) of the claimed invention are included or the phrase “consisting of” where only the specified features are included, unless otherwise stated. The present invention includes within its scope all stereoisomeric and isomeric forms of the compounds disclosed herein, including all diastereomeric isomers, racemates, enantiomers and mixtures thereof. It is also understood that the compounds of the invention may be present as E and Z isomers, also known as cis and trans isomers. Thus, the present disclosure should be understood to include, for example, E, Z, cis, trans, (R), (S), (L), (D), (+), and/or (−) forms of the compounds, as appropriate in each case. Where a structure has no specific stereoisomerism indicated, it should be understood that any and all possible isomers are encompassed. Compounds of the present invention embrace all conformational isomers. Compounds of the present invention may also exist in one or more tautomeric forms, including both single tautomers and mixtures of tautomers. Also included in the scope of the present invention are all polymorphs and crystal forms of the compounds disclosed herein.

The percentages are herein expressed by weight, unless otherwise specified.

The present invention relates to a method for labeling or detecting or targeting an eukaryotic cell from a multicellular organism, the method comprising the steps of:
a) contacting a sample comprising eukaryotic cells with at least one modified monosaccharide compound of the pentose phosphate pathway, excluding arabinose;
b) contacting the sample of step (a) with a compound bearing a first reactive group, optionally in presence of copper; and
c) optionally comprising detecting the compound of step (b) bond to the monosaccharide of step (a);
   wherein said at least one modified monosaccharide compound of the pentose phosphate pathway (excluding arabinose), comprise a reactive group X, called a second reactive group, the first and the second reactive groups are able to react together in a click chemistry reaction, as to obtain the compound of step (b) bond to the said monosaccharide. The reaction between the first and the second reactive groups allows the compound of step (b) to be bond to the modified monosaccharide compound of the pentose phosphate pathway.

Click chemistry is a well-known method from a skilled person for attaching a probe or a substrate of interest to a specific biomolecule, such as a modified monosaccharide compound according to the invention. An azide alkyne cycloaddition is a well-known so-called click chemistry reaction, in the presence or not of a copper catalyst, in which the azide group reacts with the alkyne group to afford a triazole. The alkyne group can be strained or not.

Such azide alkyne cycloaddition can be performed in copper catalyzed conditions in the presence of a ligand, preferably a tris-triazole ligand such as TGTA (Tris(1-(-D-glucopyranosyl)-1 [1,2,3]-triazol-4-yl)methyl)amine) or TBTA (Tris-[(1-benzyl-1 1,2,3-triazol-4-yl) methyl]amine). Other appropriate ligands frequently used are: tris(3-hydroxypropyl triazolylmethyl)amine (THPTA), 2-(4-((bis((1-tert-butyl-1 1,2,3-triazol-4-yl)methyl)amino)methyl)-1H-1,2,3-triazol-1-yl)ethanesulfonic acid (BTTES), tris((1-((( ethyl) carboxymethyl)-(1,2,3-triazol-4-yl)) methyl) amine, bathophenanthroline disulfonate, or tris(2-benzimidazolylmethyl)amines.

Alternatively, azide alkyne cycloaddition can be performed in the absence of copper, if a strained alkyne is used, such as azadibenzocyclooctyne (ADIBO, DIBAC or DBCO) or tetramethoxydibenzocyclooctyne (TMDIBO). Other appropriate strained alkynes frequently used for copper-free reaction include: cyclooctyne (OCT), aryl-less cyclooctyne (ALO), monofluorocyclooctyne (MOFO), difluorocyclooctyne (DIFO), dibenzocyclooctyne (DIBO), dimethoxyazacyclooctyne (DIMAC), biarylazacyclooctynone (BARAC), bicyclononyne (BCN), tetramethylthiepinium (TMTI, TMTH), difluorobenzocyclooctyne (DIFBO), oxa-dibenzocyclooctyne (ODIBO), carboxymethylmonobenzocyclooctyne (COMBO), or benzocyclononyne.

Other reactive groups and other reactions are possible for click chemistry, such as: Staudinger Ligation (first reactive group=azide and second reactive group=phosphine), copper-free click-chemistry (first reactive group=azide and second reactive group=constrained alkyne (intracyclic alkyne)), carbonyl condensation (first reactive group=aldehyde or ketone and second reactive group=hydrazide or oxyamine), thiol-ene click chemistry (first reactive group=thiol and second reactive group=alkene), nitrile-oxide-ene click chemistry (first reactive group=nitrile oxide or aldehyde, oxime, or hydroxymoyl chloride or chlororoxime and second reactive group=alkene or alkyne), nitrile imine-ene click chemistry (first reactive group=nitrile imine or aldehyde, hydrazone, or hydrazonoyl chloride or chlorohydrazone and second reactive group=alkene or alkyne), inverse electron demand Diels-Alder ligation (first reactive group=alkene and second reactive group=tetrazine), isonitrile-tetrazine click chemistry (first reactive group=isonitrile and second reactive group=tetrazine), Suzuki-Miyaura coupling (first reactive group=aryl halide and second reactive group=aryl boronate), His-tag (first reactive group=oligo-histidine and second reactive group=nickel-complex or nickel ligand).

### Monosaccharide Compounds of the Pentose Phosphate Pathway

The pentose phosphate pathway is described in many reviews, such as in Mujaji B. W., “the pentose phosphate pathway revised” in Biochemical education, 8(3) 1980, pp 76-78, or Jin and Zhou: Pentose Phosphate Pathway In Cancer—Oncology Letters 17: 4213-4221 (2019 DOI: 10.3892/01.2019.10112). The compounds of the invention are selected among the pentose phosphate pathway, excluding arabinose.

In a particular embodiment, the compounds of the pentose phosphate pathway used in the present invention are selected in the group consisting of ribose, D-ribose, L-ribose, ribose D-ribose 5-P, D-ribose 1-P, D-ribose 1,5-P, ribulose, ribulose 5-P, L-ribulose, L-ribulose 5-P, D-ribulose, D-ribulose 5-P, arabinitol, L-arabinitol, xylulose, xylulose-5-P, D-xylulose, D-xylulose-5-P, L-xylulose, xylose, D-xylose, L-xylose, and xylitol. In a more particular embodiment, the compounds of the pentose phosphate pathway used in the present invention are selected in the group consisting of ribose, D-ribose, L-ribose, ribose 5-P, D-ribose 5-P, D-ribose 1-P, D-ribose 1,5-P, ribulose, ribulose 5-P, L-ribulose, L-ribulose 5-P, D-ribulose, and D-ribulose 5-P. In another particular embodiment, the compounds of the pentose phosphate pathway used in the present invention are selected in the group consisting of ribose, D-ribose, and L-ribose. In a further embodiment, the compounds of the pentose phosphate pathway used in the present invention are selected in the group consisting of D-ribose and L-ribose. In yet another particular embodiment, the compounds of the pentose phosphate pathway used in the present invention are selected in the group consisting of xylose, D-xylose, and L-xylose. In a further particular embodiment, the compounds of the pentose phosphate pathway used in the present invention are selected in the group consisting of D-ribose and D-xylose.

According to the invention, the modified monosaccharide compound of the pentose phosphate pathway, excluding arabinose, comprises a reactive group X (second reactive group) suitable for reacting in a click chemistry reaction, preferably in an azide alkyne cycloaddition. Thus, X includes any reactive group able to react with a further reactive group by a click chemistry reaction such as reactive groups as above defined. In a particular embodiment X comprises any groups consisting in or bearing an azido group (—N₃) and groups consisting in or bearing an alkyne group (—C≡C—) strained or not. In a preferred embodiment, X is an azido group (—N₃).

According to a particular embodiment, the modified compound of the pentose phosphate pathway used in the present invention is selected from the group consisting of:

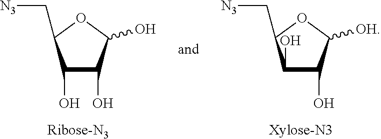

The modified monosaccharide compound of the pentose phosphate pathway can be used according to the present invention at any concentration since it does not present toxicity towards cells. According to a particular embodiment, the concentration of the monosaccharide compound of the pentose phosphate pathway can vary from 10 μM to 100 mM, preferably from 1 mM to 50 mM, more preferably from 1 mM to 20 mM.

### The Compound Bearing a First Reactive Group

The compound bearing a first reactive group comprises or is a directly detectable moiety or comprises or is an indirectly detectable moiety. Without being bound to any theory, the cells are coupled to the compound bearing a first reactive group due to the click reaction with the second reactive group of the monosaccharide of step (a) which has been assimilated by the cell membranes at step (a). The detectable moiety (or label), namely a moiety capable to be detected by techniques known by one skilled in the art, such as fluorescence, colorimetry or luminescence. The imaging techniques can thus be fluorescence, magnetic resonance or computed tomography.

According to one embodiment, said compound can comprise or can be a detectable moiety, namely a moiety consisting in or bearing a detectable substance (or a label), namely a substance capable to be detected by techniques known by one skilled in the art, such as fluorescence, colorimetry or luminescence.

According to another embodiment, said compound bearing a first reactive group comprises or is an indirectly detectable moiety which is a first ligand (or more specifically a first binding protein bearing a said first reactive group) and detection and/or immobilizing in step (c), as detailed below, can occur by contacting said eukaryotic cell coupled to said first ligand (or more specifically first binding protein) with a second ligand (or second binding protein) reacting or binding specifically to said first ligand (or more specifically first binding protein).

More particularly, said compound is a first ligand, preferably biotin, bearing a said first reactive group, and in step c) said eukaryotic cells coupled to said first ligand are detected by reaction of said eukaryotic cells with an antibody or another protein specific to said first ligand, said antibody bearing a detectable substance or moiety, preferably a fluorochrome or luminescent molecule or an enzyme.

The detectable substance or moiety can be selected among dyes, radiolabels and affinity tags. In particular, the dyes can be selected from the group consisting of fluorescent, luminescent or phosphorescent dyes, preferably dansyl, fluorescein, acridine, rhodamine, coumarin, BODIPY and cyanine dyes. More specifically, the fluorescent dyes can be selected among the dyes marketed by Thermo Fisher such as the Alexa Fluor dyes, Pacific dyes or Texas Red or by other providers for cyanines 3, 5 and 7. In particular, dyes bearing azide for CuAAC are commercially available for Alexa Fluor® 488, 55, 594 and 647 and for TAMRA (tetramethylrhodamine). In a second aspect, the detectable substance or moiety (or label) can be an affinity tag. Such an affinity tag can be for instance selected from the group consisting of biotin, His-tag, Flag-tag, strep-tag, sugars, lipids, sterols, PEG-linkers, and co-factors. In a particular embodiment, the detectable substance is a biotinylated label. Biotins linked to azide are commercially available (Biotin azide). In a preferred embodiment, the label is a fluorescent label.

According to the invention, the compound bearing a first reactive group comprises a first reactive group which is complementary with a second reactive group X as above defined for reacting together in a click chemistry reaction, preferably an azide alkyne cycloaddition. In a particular embodiment, the first reactive group of the compound comprises any group consisting in or bearing an azido group (—N₃) and groups consisting in or bearing a strained or not alkyne group (—C≡C—).

In the above-mentioned listing of groups involved in the reactions, the first reactive group and the second reactive group X can be permuted. All the above-mentioned chemical reactions result in a covalent link. For instance, when X is an azido group (—N₃) or a group bearing an azido group, then the first reactive group is an alkyne or a group bearing an alkyne group. When X is an alkyne or a group bearing an alkyne group, then the first reactive group is an azido group (—N₃) or a group bearing an azido group. In a preferred embodiment of the invention, the second reactive group X is an azido group (—N₃) and the first reactive group is an alkyne group ('C≡C—).

### Method for Labeling, Detecting or Targeting

The step a) according to the method for labeling, detecting or targeting an eukaryotic cell from a multicellular organism comprises contacting a sample comprising an eukaryotic cell with at least one modified monosaccharide compound of the pentose phosphate pathway, as defined above, comprising a reactive or a functional group X. Such contacting step a) allows the incorporation of the at least one modified monosaccharide compound in the membrane of said eukaryotic cell from a multicellular organism, more specifically on the surface of said cell. Such process may correspond to an assimilation of the at least one modified monosaccharide compound of the pentose phosphate pathway by said eukaryotic cell. Accordingly, said cell presents the at least one modified monosaccharide compound of the pentose phosphate pathway on its surface or membrane.

The step b) comprises contacting the sample of step (a) (in which the at least one modified monosaccharide compound of the pentose phosphate pathway is incorporated in the membrane of an eukaryotic cell) with a compound comprising a first reactive group as above defined. Such step b) allows to generate the click chemistry reaction between the first reactive group of the compound and the second reactive group X of the at least one modified monosaccharide compound of the pentose phosphate pathway, thereby providing a coupled eukaryotic cell from a pluricellular organism which is labelled or targeted or can be labelled or targeted thereafter (as detailed above).

A preferred embodiment of the invention is a method for labeling, detecting or targeting an eukaryotic cell from a multicellular organism, the method comprising the steps of:
a) contacting a sample comprising an eukaryotic cell with at least one compound of the pentose phosphate pathway, as defined above, and more specifically a compound selected in the group consisting of ribose, D-ribose, L-ribose, ribulose, D-ribulose, and L-ribulose; further comprising an azido group, and
b) contacting said sample with a compound comprising a first reactive group, optionally in presence of copper.

One skill in the art knows how to implement steps (a) or (b). According to a particular embodiment, said steps (a) and/or (b) are carried out in culture or incubation media allowing the growth of the sample comprising an eukaryotic cell, preferably specific to the growth of the said eukaryotic cell.

More specifically, the culture conditions (including time and cell culture medium) of steps (a) or (b) are adapted to the eukaryotic cell to be labelled, detected or targeted. The cell culture medium can be supplemented by any compound to enhance or stimulate doubling of cells and/or assimilation of the modified monosaccharide compound of the pentose phosphate pathway on the surface or membrane of the cell.

According to a particular embodiment, the duration of step (a) allows the incorporation of the at least one monosaccharide compound of the pentose phosphate in the membrane of said eukaryotic cell. More specifically, duration of step (a) is at least the doubling time of the eukaryotic cell to be labelled, detected or targeted. More particularly, duration of step (a) is less than five times the doubling time of the eukaryotic cell to be labelled, detected or targeted. According to a particular embodiment, duration of step (a) corresponds to one doubling time or two doubling times of the eukaryotic cell to be labelled, detected or targeted.

According to a particular embodiment, said steps (a) and/or (b) are carried out with reactants and/or catalysts for generating the reaction of said first reactive group with said second reactive group.

It is interesting to note that the monosaccharide compound of the pentose phosphate pathway used according to the invention presents a low or no toxicity towards cells, so that the used amount thereof can vary in a large range. Such amount will be determined by one skilled in the art so that the amount is sufficient to label, identify or detect eukaryotic cells.

The method may be implemented with any sample, typically a biological sample of a subject, e.g. a fluid, such as a sample of blood, plasma, serum, urine, cerebrospinal fluid or a sample from a tissue of a subject or a part thereof. The invention may be implemented with samples from any subject, including any human patient having or suspected to have cancer. The method is typically performed on a sample of, or derived from, blood, serum or plasma, such as a pre-treated blood sample. The sample may be treated prior to being used in the invention (e.g., diluted, concentrated, separated, partially purified, frozen, etc.). According to a particular embodiment, each sample used at step (a) of the method comprises a cell population or preferably an individual cell, preferably obtained by cell sorting, in particular by flow cytometry. When the method is implemented in vivo, the method can be implemented to the whole body of the subject or a part thereof. In that context, the monosaccharide compound used according to the invention and optionally the compound bearing a first reactive group can be administered enterally (including orally) or parenterally (including intravenously or intramuscularly).

In order to detect the coupled eukaryotic cell from a pluricellular organism, the method further includes a step c) comprising detecting the compound of step (b) bond to the monosaccharide of step (a).

Advantageously, the present invention comprises the further step (c) of detecting an eukaryotic cell in detecting whether said eukaryotic cell is coupled with the compound bearing the first reactive group of step (b) and/or in immobilizing said eukaryotic cell coupled with the compound bearing the first reactive group onto a solid substrate, wherein said compound bearing the first reactive group is a moiety or molecule comprising a detectable substance or capable to react or to be bound to a detectable substance or preferably said compound bearing the first reactive group is a first molecule being capable to react or to be bound to a second molecule and/or to a solid substrate, preferably said second molecule comprising a detectable substance and/or said second molecule being bound or capable to be bound to a said solid substrate.

Accordingly, the present invention enables labeling of eukaryotic cells from a multicellular organism as well as numbering or detecting of eukaryotic cells as well as concentrating and/or isolating eukaryotic cells, optionally immobilized on a solid support; especially with a solid support constituted of magnetic beads bearing the said first reactive group.

More particularly, said compound bearing the first reactive group is a first molecule being capable to react or to be bound to a second molecule and/or to a solid substrate, preferably said second molecule comprises a detectable substance, the method comprising the step c) of detecting eukaryotic cells in detecting whether said eukaryotic cell comprises said detectable molecule or moiety bound to said eukaryotic cell.

According to the method of the present invention, in absence of labeling or detection, one can conclude that the implemented sample does not comprise any eukaryotic cell from a pluricellular organism.

The said detecting step c) can be carried out in a liquid medium or on a solid substrate. Preferably, the method for labeling or detecting or targeting a eukaryotic cell from a multicellular organism is an in vitro method.

According to a particular embodiment, the method of the present invention can further comprise one or more washing steps.

According to a particular embodiment, the method according to the invention can be carried out with one or more samples simultaneously, using for instance a micro-well plate. The microplate typically has 6, 12, 24, 48, 96, 384 or 1536 sample wells. According to said particular embodiment, each sample well used at step (a) of the method preferably comprises a cell population or preferably an individual cell, more preferably obtained by cell sorting, in particular by flow cytometry.

A further object of the invention is thus an in vitro use of at least one modified monosaccharide compound of the pentose phosphate pathway, excluding arabinose, for labeling or detecting an eukaryotic cell from a pluricellular organism, preferably with a compound comprising a first reactive group, and optionally with a second molecule and/or to a solid substrate, preferably said second molecule comprising a detectable substance.

A further object of the invention is a kit for implementing a method for labeling or detecting an eukaryotic cell from a multicellular organism as defined herein comprising:
a modified monosaccharide compound of the pentose phosphate pathway as defined above, and
a compound bearing a first reactive group, as defined above.

According to a particular embodiment, the kit can further comprise a second molecule and/or a solid substrate, as defined above, preferably said second molecule or solid substrate comprising a detectable substance, the compound bearing the first reactive group being a first molecule capable to react or to be bound to the second molecule and/or to the solid substrate.

A further object is a use of a kit as defined above for implementing a method for labeling or detecting an eukaryotic cell from a multicellular organism as defined herein.

According to a particular embodiment, the eukaryotic cell from a multicellular organism is a cell susceptible to be a cancer or tumoral cell. Accordingly, the method and the kit according to the invention are useful to identify cancer or tumoral cells by detection of labeling.

### Method for Identifying or Isolating Cancer Cells or Diagnosing Cancer

The present invention further relates to a method, preferably an in vitro or ex vivo method, for identifying or isolating cancer cells or diagnosing a cancer in a subject comprising implementing a method for labelling or detecting an eukaryotic cell as defined herein of said subject or of a biological sample from said subject. In a preferred embodiment, the method for identifying or isolating cancer cells or for diagnosis a cancer in a subject further comprises the step of detecting the labeling and optionally comparing the labeling to a reference level.

The biological sample from a subject is as defined above, and preferably the sample is a sample from a human patient having or suspected to have cancer. According to a particular embodiment, each sample used at step (a) comprises a cell population or preferably an individual cell, preferably obtained by cell sorting, in particular by flow cytometry.

The sample is more specifically suspected to comprise cancer cells.

Examples of such samples include fluids such as blood, plasma, saliva, urine and seminal fluid samples, as well as biopsies, organs, tissues or cell samples. The sample may be treated prior to its use.

Cancer cells that are identified or isolated according to the invention can be of any type. They can come from solid tumors or hematopoietic cancers. Cancer cells include circulating or non-circulating tumor cells. Circulating tumor cells (CTCs) are cells that have shed into the vasculature or lymphatics from a primary tumor and are carried around the body in the blood circulation. CTCs constitute seeds for the subsequent growth of additional tumors (metastases) in distant organs, a mechanism that is responsible for the vast majority of cancer-related deaths.

The detection and analysis of cancer cells according to the present invention can assist early patient prognoses and determine appropriate tailored treatments. The ability to monitor the disease progression over time can facilitate appropriate modification to a patient's therapy, potentially improving their prognosis and quality of life. In case of detection and analysis of circulating tumor cells, the method can allow early detection of cancers and in particular metastases. In that respect, the sample according to the invention is a blood fluid. Blood tests are easy and safe to perform and multiple samples can be taken over time. The important aspect of the ability to prognose the future progression of the disease is elimination (at least temporarily) of the need for a surgery when the repeated CTC counts are low and not increasing; the obvious benefits of avoiding the surgery include avoiding the risk related to the innate tumor-genicity of cancer surgeries. To this end, technologies, as the present invention, with the requisite sensitivity and reproducibility to detect CTCs in patients with metastatic disease are of tremendous interest.

As used herein the expression “detecting the labelling” may include the visualizing, or detecting the presence of labeled eukaryotic cells from a multicellular organism or also the measuring of such labelling. The measuring of such labelling, such as fluorescence, allows to detect, identify or isolate cancer cells, optionally by comparing the labeling to a reference level.

It has been found herein that assimilation of the modified monosaccharide compound of the pentose phosphate pathway, in cancer cells is different from non-cancer cells, and more particularly is higher compared to non-cancer cells (e.g. reference level or control sample). For instance, the fluorescence intensity is higher for cancer cells compared to non-cancer cells (it can be so after one or two cell doubling time).

Therefore, the present invention relates to a method for identifying or isolating cancer cells or for diagnosis a cancer in a subject comprising:
implementing a method for labeling an eukaryotic cell as described herein of a sample from said subject;
detecting the labeling, after implementing of said method for labeling, and optionally comparing the labeling to a reference level; and then
identifying cancer cells or diagnosing a cancer based on the measuring of the label.

The method for identifying or isolating cancer cells or for diagnosis a cancer can be advantageously performed within one cell cycle period of time. Detection of the label is preferably performed from 10 hours to 40 hours, more preferably from 16 to 24, 25, or to 36 hours after implementing said method for labeling, and more specifically after implementing step (a) as detailed above.

The method can optionally comprise a comparison to a reference level, more specifically to a control sample or reference. The reference level can be the intensity of the labeling (such as fluorescence) measured in a normal cell (e.g. non cancer cell) and/or a known cancer cell. Preferably, the cell of reference is the closest of the cell to be studied, preferably a cell from the same cell line, same organ and/or same type. The method may comprise a previous step of providing a tumor sample and a histologically matched normal tissue from the subject.

According to specific embodiments, the cancer cells are identified or the cancer is diagnosed when the measuring of the label of the sample is higher than the measuring of the label of a control sample which is a non-cancer sample. By “higher measuring”, it denotes that the labeling ratio of the sample with respect to the non-cancer sample is more than 1.3, 1.4, 1.5, 1.6, 1.7, 1.8, 1.9, 2.0, 2.1, 2.2, 2.3, 2.4, 2.5, or 3.0. More specifically, the labeling ratio of the sample with respect to the non-cancer sample is more than 1.5, 1.6, 1.7, 1.8, 1.9, 2.0, 2.1, 2.2, 2.3, 2.4, 2.5, or 3.0.

According to a particular embodiment, the cancer to be diagnosed is chosen among rectal cancer, colorectal cancer, stomach cancer, head and neck cancer, thyroid cancer, cervical cancer, uterine cancer, breast cancer, ovarian cancer, brain cancer, lung cancer, skin cancer, bladder cancer, blood cancer, renal cancer, liver cancer, prostate cancer, multiple myeloma, and endometrial cancer. More specifically, the cancer to be diagnosed is selected from the group consisting of: bladder, blood, skin, pancreas, brain, liver, kidney, lung, muscle, lymphocyte, prostate, stomach, and breast cancer. According to a more particular embodiment, the cancer to be diagnosed is selected from the group consisting of: bladder, blood, colon, stomach, breast, lung, skin, and pancreas cancers.

According to a particular embodiment, the cancer cells to be identified or isolated are cancer cells deriving from the above listed cancers.

### Pharmaceutical Composition and Uses Thereof

The present invention relates to a composition comprising an eukaryotic cell presenting on its surface at least one modified monosaccharide compound of the pentose phosphate pathway, as above defined, operably linked or not to an anti-cancer drug or to particles comprising at least one anti-cancer drug.

In one particular aspect, the cell presenting on its surface at least one modified monosaccharide compound of the pentose phosphate pathway as above defined operably linked or not to an anti-cancer drug or to particles comprising at least one anti-cancer drug can be prepared as described above in the method of the invention.

The composition according to the invention can be prepared by the method comprising the following steps:
a) contacting a composition comprising eukaryotic cells from a multicellular organism with at least one modified monosaccharide compound of the pentose phosphate pathway as defined above;
b) contacting the composition of step (a) with a compound bearing a first reactive group and optionally comprising operably linked anticancer drugs or particles comprising at least one anti-cancer drug, optionally in presence of copper.

Said steps are similar to the ones detailed above. Step a) allows said cell to present the at least one modified monosaccharide compound of the pentose phosphate pathway on its surface or membrane. Step b) allows to generate the click chemistry reaction between the first reactive group of the compound and the second reactive group X of the at least one modified monosaccharide compound of the pentose phosphate pathway, as defined above.

According to one embodiment, the compound with the first reactive group can also comprise or be an operably linked anticancer drug or particles comprising at least one anti-cancer drug attached to the said compound, step (b) thus allows to provide eukaryotic cells from a pluricellular organism coupled with anti-cancer drugs or with particles comprising anti-cancer drugs.

The method allows to prepare, by a click chemistry reaction as above detailed, a conjugate in which a modified monosaccharide compound the pentose phosphate pathway, as defined above, is operably linked with the anti-cancer drug or particles comprising the same, and the eukaryotic cell presents on its surface said conjugate. In a particular embodiment, the conjugate is prepared by a click chemistry reaction between anti-cancer drugs comprising alkyne groups or particles presenting on their surface alkyne groups and modified monosaccharide compounds of the pentose phosphate pathway which comprise azido groups.

According to another embodiment, the compound with the first reactive group is a first ligand (or more specifically first binding protein) able to react or bind to a second ligand (or more specifically second binding protein) which is an anti-cancer agent. According to such embodiment, the method allows to prepare, by a click chemistry reaction as above detailed, a conjugate in which a modified monosaccharide compound of the pentose phosphate pathway is linked to a first ligand able to react or bind to a second ligand which is an anti-cancer agent, and the eukaryotic cell presents on its surface said conjugate. In a particular embodiment, the conjugate is prepared by a click chemistry reaction as detailed above.

As used herein an “anti-cancer drug” corresponds to any drug currently used in cancer therapy, such as an antitumoral drug. In a preferred embodiment, the anti-cancer drug is selected from the group consisting of chemotherapeutics, anti-cancer antibodies, hormonal therapy, immunotherapy, and kinase inhibitors.

The terms “operably linked anticancer drug” refers to anticancer drug which is linked, preferably covalently, while being able to present its therapeutic effect.

The particles comprising at least one anti-cancer drug are anti-cancer drug-containing particles, preferably nanoparticles, with first reactive groups as defined above. The particles can be bicyclo[6.1.0]nonyne-modified glycol chitosan nanoparticles (BCN-CNPs), for instance. CNPs are known to be able to encapsulate carious drugs with high compatibility and are widely used for drug delivery.

Chemotherapy may include an inhibitor of topoisomerases I or II, a DNA crosslinker, a DNA alkylating agent, an anti-metabolic agent and/or inhibitor of the mitotic spindles. Inhibitors of topoisomerases I and/or II include, but are not limited, to etoposide, topotecan, camptothecin, irinotecan, amsacrine, intoplicine, anthracyclines such as doxorubicine, epirubicine, daunorubicine, idanrubicine and mitoxantrone. Inhibitors of Topoisomerase I and II include, but are not limited to, intoplecin.

DNA crosslinkers include, but are not limited to, cisplatin, carboplatin and oxaliplatin.

Anti-metabolic agents block the enzymes responsible for nucleic acid synthesis or become incorporated into DNA, which produces an incorrect genetic code and leads to apoptosis. Non-exhaustive examples thereof include, without limitation, folic acid antagonists, pyrimidine analogs, purine analogs and adenosine deaminase inhibitors, and more particularly Methotrexate, Floxuridine, Cytarabine, 6-Mercaptopurine, 6-Thioguanine, Fludarabine phosphate, Pentostatine, 5-fluorouracil, gemcitabine and capecitabine.

Alkylating agents include, without limitation, nitrogen mustards, ethylenimine derivatives, alkyl sulfonates, nitrosoureas, metal salts and triazenes. Non-exhaustive examples thereof include Uracil mustard, Chlormethine, Cyclophosphamide (CYTOXAN(R)), Ifosfamide, Melphalan, Chlorambucil, Pipobroman, Triethylenemelamine, Triethylenethiophosphoramine, Busulfan, Carmustine, Lomustine, Fotemustine, cisplatin, carboplatin, oxaliplatin, thiotepa, Streptozocin, Dacarbazine, and Temozolomide.

Inhibitors of the mitotic spindles include, but are not limited to, paclitaxel, docetaxel, vinorelbine, larotaxel (also called XRP9881; Sanofi-Aventis), XRP6258 (Sanofi-Aventis), BMS-184476 (Bristol-Meyer-Squibb), BMS-188797 (Bristol-Meyer-Squibb), BMS-275183 (Bristol-Meyer-Squibb), ortataxel (also called IDN 5109, BAY 59-8862 or SB-T-101131 ; Bristol-Meyer-Squibb), RPR 109881A (Bristol-Meyer-Squibb), RPR 116258 (Bristol-Meyer-Squibb), NBT-287 (TAPESTRY), PG-paclitaxel (also called CT-2103, PPX, paclitaxel poliglumex, paclitaxel polyglutamate or Xyotax™), ABRAXANE® (also called Nab-Paclitaxel ; ABRAXIS BIOSCIENCE), Tesetaxel (also called DJ-927), IDN 5390 (INDENA), Taxoprexin (also called docosahexanoic acid-paclitaxel; PROTARGA), DHA-paclitaxel (also called Taxoprexin®), and MAC-321 (WYETH). Also see the review of Hennenfent & Govindan (2006, Annals of Oncology, 17, 735-749).

The immune checkpoint inhibitor can be selected from the group consisting of an anti-CTLA-4 (cytotoxic T lymphocyte associated protein 4) therapies such as ipilimumab, PD-1 (programmed cell death protein 1) inhibitors such as nivolumab, pembrolizumab, or BGB-A317, PDL1 (programmed cell death ligand) inhibitors such as atezolizumab, avelumab, or durvalumab, LAG-3 (Lymphocyte-activation gene 3) inhibitors such as BMS-986016, TIM-3 (T-cell immunoglobulin and mucin-domain containing-3) inhibitors, TIGIT (T cell immunoreceptor with Ig and ITIM domains) inhibitors, BLTA (B- and T-lymphocyte attenuator) inhibitors, IDO1 inhibitors such as epacadostat, or a combination thereof.

The hormonotherapy includes for instance Tamoxifen, Fareston, Arimidex, Aromasin, Femara, Zoladex/Lupron, Megace, and Halotestin.

The eukaryotic cells from a multicellular organism of the composition according to the invention are preferably isolated non-cancer cells. The cells are preferably isolated multipotent stem cells. The cells are preferably mesenchymal stem cells (MSC). MSC can be found in the whole body, preferably in adipose tissue, bone marrow, tissue supporting the organs, and also in bone, cartilage and muscle, etc. The eukaryotic cells according to the invention can be T-cells.

The cells are either allogenic or preferably autologous cells (i.e. from the subject or patient himself).

According to a particular embodiment, the composition comprises MSC presenting on their surfaces at least one modified monosaccharide compound of the pentose phostphate pathway as above defined. Such composition can be useful for instance for identifying cancer cells or tumors or diagnosing cancers by using the property of the monosaccharide to be detected by the compound comprising the first reactive group directly or indirectly as detailed above. Such composition can also be useful in therapy as to monitor hematopoietic transplants.

According to another particular embodiment, the composition comprises MSC presenting on their surfaces at least one modified monosaccharide compound of the pentose phosphate pathway as above defined, operably linked to an anticancer drug or to particles comprising at least one anti-cancer drug and attached to the said compound. Such composition can also be useful in therapy, in particular for the treatment of cancers.

The composition of the invention is preferably a pharmaceutical composition.

The pharmaceutical compositions contemplated herein include a pharmaceutically acceptable carrier in addition to the cell presenting the anti-cancer drug as detailed above. The term “pharmaceutically acceptable carrier” is meant to encompass any carrier (e.g., support, substance, solvent, etc.) which does not interfere with effectiveness of the biological activity of the cells and that is not toxic to the host to which it is administered. For example, for parental administration, the active compounds(s) may be formulated in a unit dosage form for injection in vehicles such as saline, dextrose solution, serum albumin and Ringer's solution. The pharmaceutical composition can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations suitable for parental administration conveniently comprise a sterile oily or aqueous preparation of the active ingredient which is preferably isotonic with the blood of the recipient.

The carrier must be “acceptable” in the sense of being compatible with the other ingredients of the formulations and not deleterious to the recipient thereof. The pharmaceutical compositions are advantageously applied by injection or intravenous infusion of suitable sterile solutions. Methods for the safe and effective administration of most of these chemotherapeutic agents are known to those skilled in the art. In addition, their administration is described in the standard literature.

The pharmaceutical composition of the invention can be used in cancer therapy, and more specifically in cancer cell therapy.

A preferred embodiment of the invention is a pharmaceutical composition as defined herein for use for treating a cancer as above defined.

A further preferred embodiment, is a method for treating a cancer in a subject in need thereof, comprising administering an efficient amount of a pharmaceutical composition as defined herein.

A further preferred embodiment, is a use of a pharmaceutical composition as defined herein for the manufacture of a medicament for the treatment of cancer.

### Imaging and Diagnosis

As above detailed, the modified monosaccharide compound of the pentose phosphate pathway is suitable for forming a detectable entity with a label, preferably a fluorescent label, particularly by a click chemistry reaction.

The present invention thus relates to the use of a modified monosaccharide compound of the pentose phosphate pathway as disclosed herein as a research tool for detecting an eukaryotic cell from a pluricellular organism, and more particularly to identify or isolate cancer cells. The invention further relates to a modified monosaccharide compound of the pentose phosphate pathway as disclosed herein, for medical imaging or diagnosis, preferably for diagnosis a cancer.

Further aspects and advantages of the present invention will be described in the following examples, which should be regarded as illustrative and not limiting.

### EXAMPLES

### Example 1: Synthesis of Xylose-N₃

Thin layer chromatography was performed over Merck 60 F254 with detection by UV, and/or by charring with sulphuric acid or KMnO₄ or phosphomolybdic acid solutions. Silica gel 60 40-63 Mm was used for flash column chromatography.

NMR spectra were taken on Bruker Avance 300 or 500 MHz spectrometers, using the residual protonated solvent as internal standard. Chemical shifts δ are given in parts per million (ppm) and coupling constants are reported as Hertz (Hz). Splitting patterns are designated as singlet (s), doublet (d), triplet (t), doublet of doublet (dd), doublet of doublet of doublet (ddd). Splitting patterns that could not be interpreted or easily visualized are designated as multiplet (m).

Mass spectra were taken on a Waters LCT Premier XE (ToF), with electrospray ionization in the positive (ESI+) or in the negative (ESI−) mode of detection.

IR-FT spectra were recorded on a Perkin Elmer Spectrum 100 spectrometer. Characteristic absorptions are reported in cm⁻¹.

Specific optical rotations were measured at 20° C. with an Anton Paar MCP 300 polarimeter in a 10-cm cell at 20° C. and 589 nm.

All biological and chemical reagents were of analytical or cell culture grade, obtained from commercial sources, and used without further purifications.

To a solution of D-xylose (4.00 g, 26.6 mmol, 1.0 eq.) in dry pyridine (90 mL) was added methoxyamine hydrochloride (2.72 g, 32.0 mmol, 1.2 eq.) and the mixture was stirred at room temperature for 15 hours. Solvents were removed under reduced pressure and the residue was co-evaporated with toluene three times. The residue was resuspended in 2,2-dimethoxypropane (100 mL) and 7-toluenesulfonic acid (1.01 g, 5.33 mmol, 0.2 eq.) was added and the suspension was heated to reflux for 4 hours followed by further 15 hours of stirring at room temperature. The reaction mixture was filtered over Celite® and solvents were evaporated. The residue was dissolved in ethyl acetate (200 mL) and was washed with saturated aq. NaCl solution (2×150 mL). Purification by silica flash column chromatography (cyclohexane/ethyl acetate 9:1) yields a mixture of isomers 2:3,4:5-diisopropylidene-D-xylose O-methyloxime (IIE/IIZ) and an unknown impurity (NMR ratio 6:1:0.4, 5.83 g) as colorless oil. This mixture was used without further purification in the next step. An aliquot of pure (2E) was obtained by a second flash column chromatography (dichloromethane/MTBE 98:2) and characterized.

A solution of 2:3,4:5-diisopropylidene-D-xylose O-methyloxime (II) (IIE/IIZ) and impurity (1.50 g) in 80% (v/v) aqueous acetic acid (30 mL) was heated to 40° C. at 200 mbar of pressure on a rotavap. After 2.5 hours solvents were removed under reduced pressure and the residue was co-evaporated with toluene. A mixture of isomers 2:3-isopropylidene-D-xylose O-methyloxime (IBE/111Z) (NMR ratio 4:1, 876 mg, 58% over 3 steps) were obtained after silica gel flash column chromatography (cyclohexane/ethyl acetate 1:1) as colorless oil. Purity of more than 95% by NMR.

To a solution of 2:3-isopropylidene-D-xylose O-methyloxime (III) (IIIE/IIIZ) (100 mg, 0.46 mmol, 1.0 eq.) in dry pyridine (2.0 mL) at −20° C., mesyl chloride (0.10 mL, 1.37 mmol, 3.0 eq.) was added and the reaction mixture was stirred for 1.5 hours at −20° C. After quenching the reaction with CH₃OH (0.3 mL), solvents were removed under vacuum. The resulting residue was purified by silica flash column chromatography (cyclohexane/ethyl acetate 6:4) to yield a mixture of 2:3-isopropylidene-5-O-methanesulfonyl-D-xylose O-methyloxime (IVE/IVZ) (NMR ratio 4:1, 110 mg, 81%) as colorless oil. An aliquot of pure (IVE) isomer was obtained by flash column chromatography (dichloromethane/diethyl ether 9:1) and characterized. Purity of more than 95% by NMR.

To a solution of (IVE/IVZ) (810 mg, 2.72 mmol, 1.0 eq.) in N,N-dimethylformamide (30.0 mL, 0.10 M), sodium azide (531 mg, 8.17 mmol, 3.0 eq.) was added and the reaction mixture was heated at 80° C. for 15 hours. Solvent was then removed under reduced pressure and the residue was purified by flash column chromatography (cyclohexane/ethyl acetate 9:1) to yield a mixture of 5-azido-5-deoxy-2:3-isopropylidene-D-xylose O-methyloxime (VE/VZ) (NMR ratio 7:3, 637 mg, 96%) as yellowish oil. A fraction of (VE) was isolated by flash column chromatography (dichloromethane/MTBE 97:3) for its characterizations. Purity of more than 95% by NMR.

To a solution of (VE/VZ) (820 mg, 3.36 mmol, 1.0 eq.) in 80% (v/v) aqueous acetic acid (120 mL), formaldehyde (0.8 mL) was added and the reaction mixture was stirred for 1 hour at room temperature. Solvents were removed under reduced pressure and co-evaporation with toluene was done to assure complete elimination of acetic acid. The crude compound 5-azido-5-deoxy-2:3-isopropylidene-D-xylose (VI) (682 mg) was obtained.

A solution of 5-azido-5-deoxy-2:3-isopropylidene-D-xylose (VI) (100 mg) in a mixture of AcOH/H₂O (8 mL:5 mL) was stirred at 100° C. for 1 hour. The reaction medium was concentrated and coevaporated 5 times with toluene to result in a brown oil.

The crude residue was purified with silica flash column chromatography (SiO2, 12 g, solid residue, dichloromethane/methanol; 100:0 to 50:50) to obtain 5-azido-5-deoxy-xylose (xylose-N3) as a colorless oil. Purity of more than 85% by NMR.

### Example 2: Synthesis of Ribose-N₃

A solution of D-ribose (10.0 g, 65.9 mmol, 1.0 eq.) in pyridine (66 mL) was heated at 100° C. for 2 hours. After cooling to room temperature, tosyl chloride (1.1 eq, 13.8 g, 72.5 mmol) was added and the reaction medium was stirred during 27 hours. Acetic anhydride (5.33 eq., 33 mL, 351 mmol) was added and the reaction medium was stirred during 43 hours before concentration under vacuum, then coevaporated with toluene to result in a black oil. The residue was resuspended in DMF (164 mL) under argon atmosphere, and sodium azide was then added (2 eq., 8.57 g, 131 mmol). The suspension was heated at 80° C. for 19 hours. Water was added, and the aqueous phase was extracted with AcOEt. Combined organic phases were washed twice with water, twice with a saturated NaCl solution, dried on Na₂SO₄, filtrated and concentrated to give 16.6 g of a black oil.

The crude product was purified with silica flash column chromatography (SiO2, liquid deposit in CH₂Cl₂, Cyclohexane/AcOEt; 95:5 to 70:30) to give two fractions of compound (2b) (2.28 g).

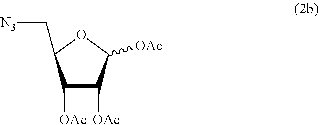

A solution of MeONa (0.1 eq., 5.4 M in MeOH, 37 μL, 0.199 mmol) was added to a solution of compound (2b) (1 eq., 600 mg, 1.99 mmol). The reaction medium was stirred 1 hour at room temperature, and neutralized to pH 5 with addition of embelite IR-120[H⁺] resin. The mixture was filtrated on cotton and concentrations under low pressure. The product was purified with silica flash column chromatography (solid deposit, CH₂Cl₂/MeOH 100:0 to 50:50) to give ribose-N3 as a colourless oil. Two batches were prepared according to the same procedure, were solubilized in water and lyophilised.

### Example 3: Labelling of Cells

### Material and Methods

Cell Culture:

The cell lines were grown in suitable medium, supplemented by 10% fetal bovine serum (FBS) (VWR international S.A.S). The culture medium was changed every two days. The passaging of cells was performed using Tryp-LE express 1× (Gibco). The cell viability was estimated using trypan blue exclusion tests.

Cell Exposure to Xylose-N₃ or Ribose-N₃ Probe:

Cells were seeded in 24-well plates, at a density of 5×10⁴ cells/well. Then, cells were incubated at 37° C., 5% CO₂, during 24 hours. The culture media were adapted to the tested cells and supplemented by 10% fetal bovine serum (FBS). The incubation time corresponded to one doubling time of the tested cells. The fluorescence signal cancer/non cancer ratios were calculated based on fluorescence intensities obtained with Xylose-N₃ or Ribose-N₃ tested on cancer and corresponding non cancer cells.

Labeling with Anti-Biotin Antibodies:

The fluorescence assimilation of Xylose-N₃ or Ribose-N₃ probes was visualized by copper-free click chemistry using sulfo-DBCO-biotin (1 mM), followed by labeling with a mouse anti-biotin Alexa Fluor 488 antibody conjugate (0.62 mg/ml stock, Jackson ImmunoResearch, dilution 1/10). The click reaction was carry-out as follows: At 24 h incubation times, the culture mediums were removed, and attached cells were washed twice with PBS. Cells were detached with Tryp-LE, washed twice with PBS, and centrifuged at 380 g for 2 min. The cellular pellets were resuspended with 10 μL of sulfo-DBCO-biotin and incubated 30 min in the dark, and at 37C°. Then, cells were washed twice with PBS, centrifuged, and cellular pellets were resuspended with 10 μL of a mouse anti-biotin antibody solution, before their incubation at room temperature in the dark. Thereafter, cells were washed twice with PBS and small cellular suspension spots (5 μL) were put in polysine® adhesion slides (VWR international S.A.S), in dark room, until dry (20 min). The spots were fixed with 4% paraformaldehyde during 20 min, in dark and room temperature. Slides were then washed twice with PBS, before recovering with square cover glasses (VWR international S.A.S) using glycerol Mounting Medium (Dako) and stored at 4° C. in a dark room.

Fluorescence Microscopy:

The fluorescence acquisition was recorded using Olympus Microscopy IX83 (Olympus Life Science) equipped with a 60×/1.3 digital opening/1.4 SRI (silicone refractive index) objective, and Hamamatsu oreca flash 4 LT camera with 109 nm/pixel calibrated pixel size. The excitation light was emitted by X-CITE 120LED using the AT180/30× excited filter and signal was monitored using AT535/40 m emission filter. Green light exposition time was 600 ms and white light exposition time was determined by DIC methods at 340 ms. The acquisition area size analyzed by the Cellsens dimension V1.16 software was different depending on the cell number containing in the spot.

Image Processing and Fluorescence Quantification:

The images were processed using count and measure unit of the Cellsens dimension V1.16 software. The background noise was deleted as follows: ROI were first defined on the background of the image and the average pixel intensity was calculated. The value thus obtained was subtracted from all pixels of the image. The fluorescence intensity of each cell was determined from a threshold set at a value of 3030. All pixels that have intensity below 3030 were not counted.

Results:

The results are presented below (Table 1) and show higher fluorescence intensities for the tested cancer cell lines in comparison to the corresponding non-cancer cell lines (ratio higher than 1).

| Human | | | | | | | Xylose-N₃ | Ribose-N₃ |
|---|---|---|---|---|---|---|---|---|
| Cancer | | | Doubling | Human non | | Doubling | Cancer/Non | Cancer/Non |
| cell Line | Organ | Disease | time | Cancer cell line | Organ | time | Cancer Ratio | Cancer Ratio |
| THP1 | Blood | monocytic acute leukemia | 26 h | Monocytes/Macr | Blood | 26 h | 25 | 20 |
| | | | | ophages | | | | |
| KCL22 | Blood | chronic myelogenous leukemia | 24 h | Lymphocytes | Blood | 24 h | 7 | 12 |
| A431 | Skin | Epidermoid carcinoma | 30 h | Hacat | Skin | 30 h | 12 | 9 |
| PANC | Pancreas | Pancreatic carcinoma | 32 h | HPNE | Pancreas | 32 h | 3 | 7 |
| Kasumi-1 | Blood | Acute Myeloid Leukemia | 48 h | B12-F8 | Blood | 24 h | 2 | 3 |
| K562 | Blood | Chronic Myeloid Leukemia | 24 h | Lymphocytes T | Blood | 24 h | 5 | 4 |
| HT29 | colon | Colorectal adenocarcinoma | 40 h | Colon epithelial | colon | 40 h | 8 | 5 |
| | | | | cell | | | | |
| AGS | Stomach | Gastric adenocarcinoma | 30 h | Stomach epitelial | Stomach | 30 h | 2 | 2 |
| | | | | cell | | | | |
| MDA- | Breast | Breast adenocarcinoma | 40 h | Breast epithelial | Breast | 40 h | 4 | 7 |
| MB-231 | | | | cell | | | | |
| A549 | Lung | Carcinoma | 40 h | Bronchial | Lung | 40 h | 10 | 7 |
| | | | | epithelial cell | | | | |
| BL-2 | Blood | non-endemic Burkitt | 24 h | Isolated primary | Blood | | 2 | 4 |
| | | lymphoma | | B cells | | | | |
| DOHH-2 | Blood | Diffuse large B-cell lymphoma | 40-48 h | Isolated primary | Blood | | 12 | 15 |
| | | germinal center B-cell type | | B cells | | | | |
| OCI-Ly3 | Blood | Diffuse large B-cell lymphoma | 24 h | Isolated primary | Blood | | 9 | 5 |
| | | | | B cells | | | | |
| MDA | Breast | Breast cancer | 30 h | HME-1 | Breast | 30 h | 3 | 8 |
| MB175 | | | | | | | | |

1. An in vitro method for labeling or detecting or targeting an eukaryotic cell from a multicellular organism, the method comprising the steps of:
a) contacting a sample comprising eukaryotic cells with at least one modified monosaccharide compound of the pentose phosphate pathway, excluding arabinose;
b) contacting the sample of step (a) with a compound bearing a first reactive group, optionally in presence of copper; and
c) optionally detecting the compound of step (b) bond to the monosaccharide of step (a), as to detect eukaryotic cells;
wherein said at least one modified monosaccharide compound of the pentose phosphate pathway comprise a reactive group X, called a second reactive group, and wherein the first and the second reactive groups are able to react together in a click chemistry reaction, as to obtain the compound of step (b) bond to the said monosaccharide.

2. The method according to claim 1, wherein the second reactive group X is an azido group (—N₃) and the first reactive group is an alkyne group.

3. The method according to claim 1, wherein said at least one modified monosaccharide compound of the pentose phosphate pathway is selected from the group consisting of ribose, D-ribose, L-ribose, ribose 5-P, D-ribose 5-P, D-ribose 1-P, D-ribose 1,5-P, ribulose, ribulose 5-P, L-ribulose, L-ribulose 5-P, D-ribulose, D-ribulose 5-P, arabinitol, L-arabinitolxylulose, xylulose-5-P, D-xylulose, D-xylulose-5-P, L-xylulose, xylose, D-xylose, L-xylose, and xylitol.

4. The method according to claim 1, wherein said at least one modified monosaccharide compound of the pentose phosphate pathway is selected from the group consisting of ribose, D-ribose, L-ribose, ribose 5-P, D-ribose 5-P, D-ribose 1-P, D-ribose 1,5-P, ribulose, ribulose 5-P, L-ribulose, L-ribulose 5-P, D-ribulose, and D-ribulose 5-P.

5. An in vitro or ex vivo method for identifying or isolating cancer cells in a subject, wherein said method comprises labeling an eukaryotic cell of a sample of the subject according to the method of claim 1.

6. An in vitro or ex vivo method for diagnosing a cancer in a subject, wherein said method comprises labeling an eukaryotic cell of a sample of the subject according to the method of claim 1.

7. The method according to claim 5, wherein the method further comprises the step of detecting the labeling, and optionally comparing the labeling to a reference level; and then identifying cancer cells based on the measuring of the label.

8. The method according to claim 5, wherein the sample is a biological sample from a subject having or being suspected to have cancer.

9. A kit for implementing the method for labeling or detecting or targeting an eukaryotic cell of claim 1 comprising:
a modified monosaccharide compound of the pentose phosphate pathway, as defined in claim 1, and
a compound bearing a first reactive group.

10. A pharmaceutical composition comprising an eukaryotic cell presenting on its surface at least one modified monosaccharide compound of the pentose phosphate pathway, as defined in claim 1, optionally linked operably or not to an anti-cancer drug or to particles comprising an anti-cancer drug.

11. The pharmaceutical composition according to claim 10, wherein the anti-cancer drug is an antitumoral drug selected from the group consisting of chemotherapeutics, anti-cancer antibodies, hormonal therapy, immunotherapy, and kinase inhibitors.

12. The pharmaceutical composition according to claim 10, wherein the eukaryotic cell is an isolated non-cancer cell.

13. A method for treating cancer, comprising administering to a subject the pharmaceutical composition according to claim 12.

14. A method for diagnosing cancer, comprising administering to a subject the pharmaceutical composition according to claim 12.

15. The method according to claim 6, wherein the cancer is chosen among rectal cancer, colorectal cancer, stomach cancer, head and neck cancer, thyroid cancer, cervical cancer, uterine cancer, breast cancer, ovarian cancer, brain cancer, lung cancer, skin cancer, bladder cancer, blood cancer, renal cancer, liver cancer, prostate cancer, multiple myeloma, and endometrial cancer.

16. A method for medical imaging or diagnosis, comprising administering to a subject a modified monosaccharide compound or a precursor thereof as defined in claim 1.

17. The method according to claim 6, wherein the method further comprises the step of detecting the labeling, and optionally comparing the labeling to a reference level; and then diagnosing a cancer based on the measuring of the label.

18. The method according to claim 6, wherein the sample is a biological sample from a subject having or being suspected to have cancer.

19. The method according to claim 13, wherein the cancer is chosen among rectal cancer, colorectal cancer, stomach cancer, head and neck cancer, thyroid cancer, cervical cancer, uterine cancer, breast cancer, ovarian cancer, brain cancer, lung cancer, skin cancer, bladder cancer, blood cancer, renal cancer, liver cancer, prostate cancer, multiple myeloma, and endometrial cancer.

20. The pharmaceutical composition according to claim 12, wherein the isolated non-cancer cell is a mesenchymal stem cell.
